(19) United States
(12) Patent Application Publication
(10) Pub. No.: US 2026/0207327 A1
(43) Pub. Date: Jul. 23, 2026

(54) A PROSTHESIS CONFIGURED TO PREVENT OR RESTRICT BILLOWING

(71) Applicant: Medtronic, Inc., Minneapolis, MN (US)

(72) Inventor: Karl L. OLNEY, Irvine, CA (US)

(21) Appl. No.: 19/139,983

(22) PCT Filed: Dec. 15, 2023
(86) PCT No.: PCT/IB2023/062796

§ 371 Date: Jun. 17, 2025

Related U.S. Application Data
(60) Provisional application No. 63/434,189, filed on Dec. 21, 2022.

(51) Int. Cl. A61F 2/24; A61F 2/91

(52) U.S. Cl. CPC A61F 2/2418; A61F 2/91; A61F 2210/0014; A61F 2250/001

(57) ABSTRACT

A prosthesis is provided having a radially expanded configuration and a radially compressed configuration. The prosthesis includes a frame comprising a plurality of side openings, a skirt coupled to the frame and at least partially covering at least one side opening of the plurality of side openings, and a skirt-reinforcement arrangement configured to prevent or restrict billowing of an area of the skirt extending over the at least one side opening. The skirt-reinforcement arrangement comprises at least one reinforcement member embedded within the skirt.

This application claims the benefit of U.S. Provisional Ser. No. 63/434,189, filed Dec. 21, 2022, the entire content of which is incorporated herein by reference.

### FIELD

The present teachings relate to a prosthesis, and in particular to a valve prosthesis.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

### Known Heart Valve Prosthesis

The present disclosure relates to improvements in a heart valve prosthesis to ensure that the heart valve prosthesis has a low profile for transcatheter delivery through a patient's vasculature.

### SUMMARY

According to a first aspect there is provided, a prosthesis having a radially expanded configuration and a radially compressed configuration, the prosthesis comprising: a frame comprising a plurality of side openings; a skirt coupled to the frame and at least partially covering at least one side opening of the plurality of side openings; and a skirt-reinforcement arrangement configured to prevent or restrict billowing of an area of the skirt extending over the at least one side opening, wherein the skirt-reinforcement arrangement comprises at least one reinforcement member embedded within the skirt.

The skirt-reinforcing arrangement is configured to prevent or restrict billowing of the skirt material that spans across the side opening of the frame of the valve prosthesis, as such billowing may undesirably result in contact between the skirt and the leaflets of the valve prosthesis after the valve prosthesis is deployed in situ. If the leaflets of the valve prosthesis contact the skirt during opening and closing in situ, such contact may cause early leaflet tissue abrasion as well as early skirt abrasion due to the undesired billowing of the skirt. Additionally, the greater relative motion between the skirt and the frame may further induce early skirt abrasion. Early leaflet tissue abrasion or early skirt abrasion has a negative impact on the long-term durability of the valve prosthesis.

This reduction in billowing of the skirt may enable a reduction in the number of frame struts needed to be used on the frame, which results in a lower profile of the prosthesis when in the radially compressed configuration for transcatheter delivery through a patient's vasculature.

In some embodiments, the skirt may be substantially tubular, and the at least one reinforcement member may extend around a minority or majority of the skirt, for example around substantially all of the skirt. For example, the at least one reinforcement member may spiral around all or part of the skirt or form bands, dashes, criss-crosses, wave patterns, or other patterns around all or part of the skirt.

In some embodiments, the frame may comprise a row of side openings extending around a perimeter thereof, and the at least one reinforcement member may extend across a plurality of side openings in the row of side openings. The at least one reinforcement member may at least partially extend across each of the plurality of side openings in the row of side openings in any direction or combination of directions (e.g., latitudinally, longitudinally, or in a wave formation).

In some embodiments, the at least one reinforcement member may extend circumferentially or around all or a portion of the skirt. For example, in some embodiments, opposite ends of the reinforcement member may be connected together to form a circumferential reinforcing ring extending around an entirety of the skirt. In some embodiments, opposite ends or edges of the reinforcement member may be connected together via crimping. In some embodiments, the skirt-reinforcement arrangement may comprise a plurality of spaced apart circumferential reinforcing rings.

In some embodiments, the skirt-reinforcing arrangement may comprise an array of overlapping reinforcement members arranged to form a reinforcing mesh.

In some embodiments, the skirt may comprise a fabric material. In some embodiments, the skirt may be formed as a braided fabric comprising a braid of a polymeric skirt material and a plurality of reinforcement members.

In some embodiments, the fabric material may comprise a polymeric skirt material. For example, in some embodiments, the polymeric skirt material may comprise one or more of PET, PTFE, and/or polyester.

In some embodiments, the at least one reinforcement member may comprise a shape memory material. For example, in some embodiments, the shape memory material may comprise nitinol.

In some embodiments, the at least one reinforcement member may be configured to have a higher rigidity than a material forming the skirt.

In some embodiments, the reinforcement member may comprise one or more of: stainless steel, PET, PTFE, nitinol cobalt-chromium alloy, ceramic, polyvinylchloride (PVC), polyethylene (PE), polypropylene (PP), polymethylmethacrylate (PMMA), titanium, and/or polyester material(s), or any other biocompatible material(s) or combination of the above or other biocompatible materials, such as a hybrid PET+nitinol wire material.

In some embodiments, the skirt may comprise a coating of a surface coating material. In some embodiments, the surface coating material may comprise PTFE. In some embodiments, the surface coating material may comprise a polymeric material. In some embodiments, the surface coating material may comprise one or more of: PTFE; polyurethane; silicone; and/or hydrogels.

In some embodiments, the at least one side opening of the plurality of side openings may be substantially diamond or hexagonal shaped. In some embodiments, each of the plurality of side openings may be substantially diamond or hexagonal shaped.

In some embodiments, the frame may comprise a row of side openings extending around a perimeter thereof. In some embodiments, the row may comprise fewer than twelve side openings, optionally less than ten side openings, for example in the range four to ten side openings, for example in the range six to nine side openings.

In some embodiments, the frame may comprise a plurality of crowns and a plurality of struts with each crown being formed between a pair of opposing struts, and wherein the plurality of side openings are defined by the plurality of crowns and the plurality of struts.

In some embodiments, the prosthesis may comprise an outer skirt coupled to the outer frame. In some embodiments, the skirt may be coupled to an inner surface or an outer surface of the frame, and the skirt may extend over fewer than all of the plurality of side openings.

In some embodiments, the frame may be formed from a self-expanding material. In some embodiments, the self-expanding material may comprise a shape memory material, for example nitinol.

In some embodiments, the prosthesis may be a heart valve prosthesis comprising a prosthetic valve component disposed within and secured to the frame, the prosthetic valve being configured to block blood flow in one direction to regulate blood flow through a central lumen of the frame. In some embodiments, the heart valve prosthesis may be configured for placement within a mitral heart valve or tricuspid heart valve in situ.

In some embodiments, the frame may comprise an inner frame and an outer frame, and wherein the plurality of side openings may comprise a plurality of side openings on the inner frame. In some embodiments, the outer frame may be connected to the inner frame and radially surround the inner frame, when the prosthesis is in the radially expanded configuration.

In some embodiments, the plurality of side openings may comprise a plurality of side openings on the outer frame

### BRIEF DESCRIPTION OF THE DRAWINGS

Embodiments will now be described with reference to the accompanying drawings, in which:

FIG. 1 is a perspective view of a prosthesis in accordance with an embodiment of the disclosure;

FIG. 2 is an atrial or inflow end view of the prosthesis shown in FIG. 1; FIG. 3 is a ventricular or outflow end view of the prosthesis shown in FIG. 1;

FIG. 4 is a perspective view of an inner frame of the prosthesis of FIG. 1 with a prosthetic valve component secured therein;

FIG. 5 is an enlarged side view of a side opening of the inner frame of FIG. 2;

FIG. 6 is a perspective atrial end view of the prosthesis shown in FIG. 1;

FIG. 7 is an atrial end view of the prosthesis shown in FIG. 1;

FIG. 8 is a side view of the inner frame of FIG. 2; and

FIG. 9 is a side view of the inner frame of FIG. 2.

### DETAILED DESCRIPTION OF CERTAIN EMBODIMENTS

The human heart includes right and left atriums, and right and left ventricles. Disposed between the left atrium and left ventricle is the mitral valve. As left atrial pressure increases above that of left ventricle, the mitral valve opens and blood passes into the left ventricle. The mitral valve is formed from a pair of leaflets having distal edges that meet so as to close the mitral valve. Each leaflet is attached to an annular region of the heart structure known as the valve annulus. Disposed between the right atrium and the right ventricle is the tricuspid valve. As right atrial pressure increases above that of right ventricle, the tricuspid valve opens and blood passes into right ventricle. The tricuspid valve is formed from three leaflets having distal edges that meet so as to close the tricuspid valve. Each leaflet is attached to an annular region of the heart structure known as the valve annulus. In addition to mitral valve and tricuspid valve, the heart includes the aortic valve and the pulmonary valve. The aortic valve permits one-way flow of blood from the left ventricle to the aorta. The pulmonary valve permits one-way flow of blood from right ventricle to pulmonary artery. Each of the aortic valve and the pulmonary valve are formed from three leaflets having distal edges that meet so as to close the respective valve. Each leaflet is attached to an annular region of the heart structure known as the valve annulus. The present teachings relate to a heart valve prosthesis for replacing diseased and/or damaged heart valves. In particular, the teachings relate to a skirt-reinforcement member for supporting or reinforcing a skirt that spans across a side opening of a frame of the prosthesis, e.g. the heart valve prosthesis.

The illustrated embodiments relate to a transcatheter valve prosthesis in the form of a heart valve prosthesis configured for placement within a mitral heart valve or a tricuspid heart valve. In alternative embodiments, skirt-reinforcement members described herein may be utilized with a transcatheter heart valve configured for placement within a pulmonary, aortic, mitral, or tricuspid valve, or may be utilized with a transcatheter valve prosthesis configured for placement within a venous valve or within other body passageways where it is deemed useful. It will be understood that the embodiments of skirt-reinforcement members described herein may be utilized with any prosthesis having a frame and a skirt for which reinforcement thereof is desirable to limit billowing and is not intended to be limited to a prothesis having an inner frame and an outer frame coupled to and radially surrounding the inner frame.

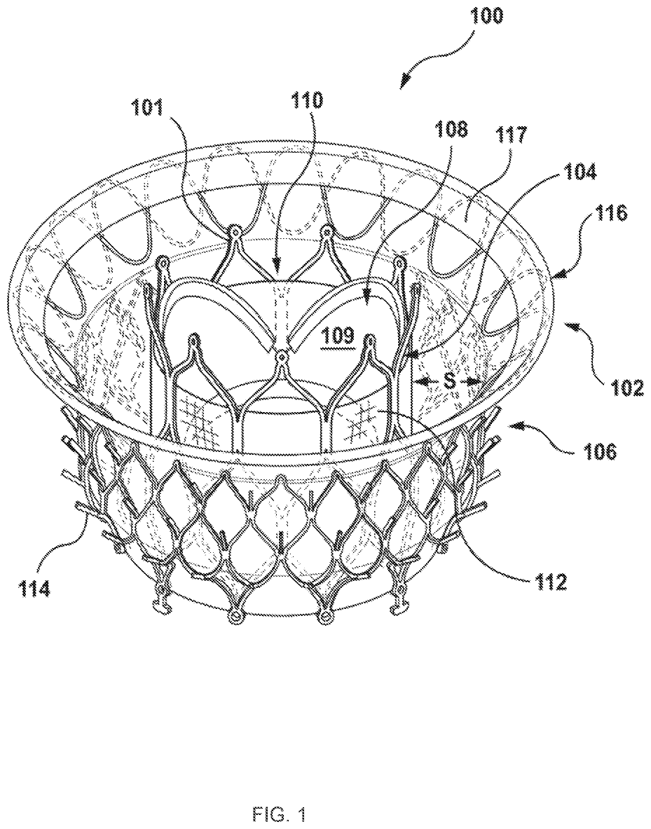

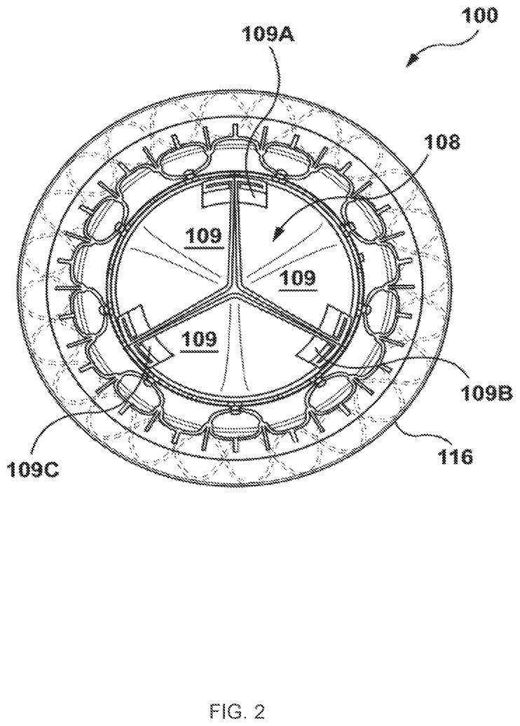

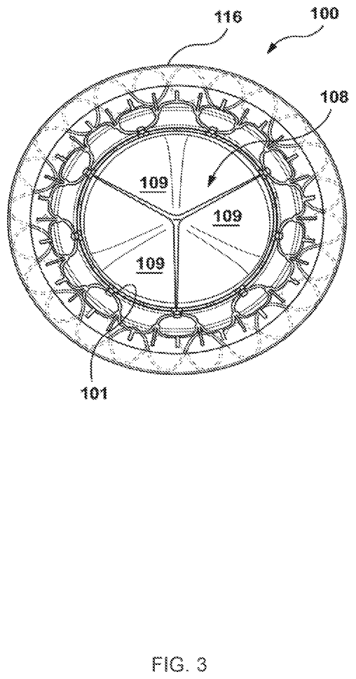

Referring to FIGS. 1 to 3, a transcatheter valve prosthesis is illustrated and is indicated generally at 100. The transcatheter valve prosthesis 100 is configured to be radially compressed into a reduced-diameter crimped configuration for delivery within a vasculature (not shown) and to return to an expanded, deployed configuration, as is illustrated in Figure FIG. 1. Stated another way, the transcatheter valve prosthesis 100 has a crimped configuration for delivery within a vasculature and an expanded configuration for deployment within a native heart valve. When in the crimped configuration, the transcatheter valve prosthesis 100 has a low profile suitable for delivery to and deployment within a native heart valve via a suitable delivery catheter that may be tracked to the deployment site of the native heart valve of a heart via any one of a transseptal, retrograde, or transapical approach.

The transcatheter valve prosthesis 100 includes a stent or frame 102. The frame 102 may be considered to be a dual frame. The frame 102 includes a valve support or inner frame 104. The frame 102 includes an anchor element or outer frame 106. The inner frame 104 is at least partially surrounded (e.g. radially) by and coupled to the outer frame 106, when the prosthesis 100 is in the radially expanded configuration. The prosthesis 100 includes a prosthetic valve component 108 including at least one leaflet disposed within and secured to the frame 102.

In the embodiment illustrated, the outer frame 106 is coupled or connected to the inner frame 104 at or near to the outflow end 103 of the inner frame 104. It will be appreciated that in alternative embodiments, the outer frame 106 may be connected to the inner frame 104 at any suitable location, such as at or near the inflow end 101 of the inner frame 104 at a point between the inflow and outflow ends 101, 103. In some embodiments, for example, the frame 102 may include an outer frame 106 that is defined by a portion of the frame 104 that is inverted so as to radially surround the inner frame 104. Put another way, the outer frame 106 may extend from an end of the inner frame 104 and be folded over so as to radially surround the inner frame 104 (e.g. such that the inner frame 104 is at least partially positioned within the outer frame 106). It will be appreciated that in such embodiments, the outer frame 106 may extend from the inflow end 101 of the inner frame 104 or the outflow end 103 of the inner frame 104.

The inner frame 104 may be tubular or cylindrical shape. The inner frame 104 defines a central lumen 110 from an inflow end 101 to an outflow end 103 thereof. The inner frame 104 is configured to support the prosthetic valve component 108 therein, which will be described in more detail below. In an embodiment, the outflow end 103 has a diameter that is substantially the same as a diameter of the inflow end 101.

The prosthesis 100 includes an inner skirt 112. The inner skirt 112 is coupled to the inner frame 104. In the embodiment shown, the inner skirt 112 is coupled to an inner surface of the inner frame 104 to line a portion thereof. Alternatively, the inner skirt 112 may be coupled to an outer surface of the inner frame 104. The inner skirt 112 may be a natural or biological material such as pericardium or another membranous tissue such as intestinal submucosa. The inner skirt may be formed from a polymeric material. The inner skirt 112 may be a fabric, for example a low-porosity woven or braided fabric. Examples of such materials are polyethylene terephthalate (PET), polyester, Dacron fabric, or PTFE. The inner skirt 112 creates fluid boundaries of a one-way fluid passage when attached to the inner frame 104 and combined with a valve. In one embodiment, the inner skirt 112 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. In some embodiments, the inner skirt 112 may be provided with a coating of a surface coating material. Put another way, the inner skirt 112 may have a layer of a coating material on a surface thereon. The surface coating material may comprise a polymeric material. The surface coating material may comprise one or more of: PTFE; polyurethane; silicone; and hydrogels.

The outer frame 106 may be tubular or cylindrical shape. The outer frame 106 functions as an anchor for the transcatheter valve prosthesis 100 to secure its deployed position within a native annulus. The outer frame 106 is configured to engage heart tissue at or below an annulus of a native heart valve, such as an annulus of a native mitral valve or native tricuspid valve. At the inflow end 101 of the inner frame 104, the outer frame 106 is radially spaced a distance S from the inner frame 104 to mechanically isolate the inflow end 101 of the inner frame 104 from the outer frame 106. The outer frame 106 includes one or more cleats or prongs 114 that extend outward from an exterior side thereof to engage heart tissue. In another embodiment, the outer frame 106 may employ barbs, spikes, or other tissue fixation mechanisms for engaging heart tissue. In some embodiments, in cross-section, or viewed along its longitudinal axis, the outer frame 106 may be D-shaped, cylindrical, elliptical, or irregularly shaped. The outer stent may also have an angled or irregular side profile. For example, the outflow end of the outer frame 106 may be angled across one or both of its proximal or distal edges. This can be useful, for example, in that it can provide additional frame-profile options that may reduce obstruction of surrounding anatomy, such as the left or right ventricular outflow tracts (LVOT and RVOT, respectively) if they prosthetic valve is implanted in either the mitral or tricuspid annulus.

The inner frame 104 and/or the outer frame 106 may be formed from a self-expanding material, for example a shape memory material. The shape memory material may be a shape memory polymer, or a shape memory alloy, such as Nitinol™.

Any portion of the frame 102 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials.

A suitable biocompatible material would be selected to provide the transcatheter heart valve prothesis 100 to be configured to be compressed into a reduced-diameter crimped configuration for transcatheter delivery to a native valve, whereby release from a delivery catheter returns the prosthesis to an expanded, deployed configuration.

The prosthesis 100 in this embodiment includes an optional brim or rim element 116. The example brim 116 shown here extends outwardly from an upstream end of the outer frame 106. The brim element 116 includes a brim frame. In the embodiment shown, the brim frame includes overlapping, 180° out of phase sinusoidal wire forms, although it will be appreciated that any suitable brim frame may be used in embodiments that include the optional brim. The brim frame is attached to the outer frame 106 by an outer skirt 117. The outer skirt 117 may be any suitable biocompatible material, for example a low-profile fabric used in bioprosthetic implants, such as woven polyethylene terephthalate (PET) fabric. The brim element 116 may act as an atrial retainer, if present. It may also aid in visualizing the prosthesis and its placement or orientation during the implant procedure under fluoroscopy, ultrasound, or another appropriate live-visualization technique. In order to provide this function, the optional brim element 116 may be configured to engage tissue above or on a native annulus, such as a supra-annular surface or some other tissue in the atrium, to thereby provide a feature for visualization, inhibit downstream migration of a prosthetic heart valve 100, assist with sealing to prevent regurgitation, or any combination of these benefits.

The prosthetic valve component 108 of the transcatheter valve prosthesis 100 is capable of regulating flow therethrough via valve leaflets that may form a replacement valve. In the illustrated embodiment, the valve component 108 is shown has having three leaflets, although other numbers of leaflets, such as in a single leaflet or bicuspid leaflet configuration, may alternatively be used. When deployed in situ, the prosthetic valve component 108 in a closed state is configured to block blood flow in one direction to regulate blood flow through a lumen 110 of the inner frame 104.

The prosthetic valve component 108 includes valve leaflets 109, e.g., three valve leaflets 109. The valve leaflets 109 are disposed to coapt within an upstream portion of the inner frame 104. Leaflet commissures 109A, 109B, 109C of the valve leaflets 109 are secured within a downstream portion of the inner frame 104, such that the valve leaflets 109 open during diastole. Leaflets 109 are attached along their bases to the inner frame 104, for example, using sutures or a suitable biocompatible adhesive. Adjoining pairs of leaflets 109 are attached to one another at their lateral ends to form leaflet commissures 109A, 109B, 109C. The orientation of the leaflets 109 within the inner frame 104 depends upon on which end of the transcatheter valve prosthesis 100 is the inflow end and which end of the transcatheter valve prosthesis 100 is the outflow end, thereby ensuring one-way flow of blood through the transcatheter valve prosthesis 100.

In some embodiments, the valve leaflets 109 may be attached to the inner skirt 112. The valve leaflets 109 may be formed of various flexible materials including, but not limited to natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

For delivery, the transcatheter valve prosthesis 100 is radially compressed into a reduced-diameter crimped configuration onto a delivery system for delivery within a vasculature. As known in the art, the delivery system includes an inner shaft that receives the transcatheter valve prosthesis 100 on a distal portion thereof and an outer sheath or capsule that is configured to compressively retain the transcatheter valve prosthesis 100 on the distal portion of the inner shaft during delivery. Stated another way, the outer sheath or capsule surrounds and constrains the transcatheter valve prosthesis 100 in the radially compressed or crimped configuration.

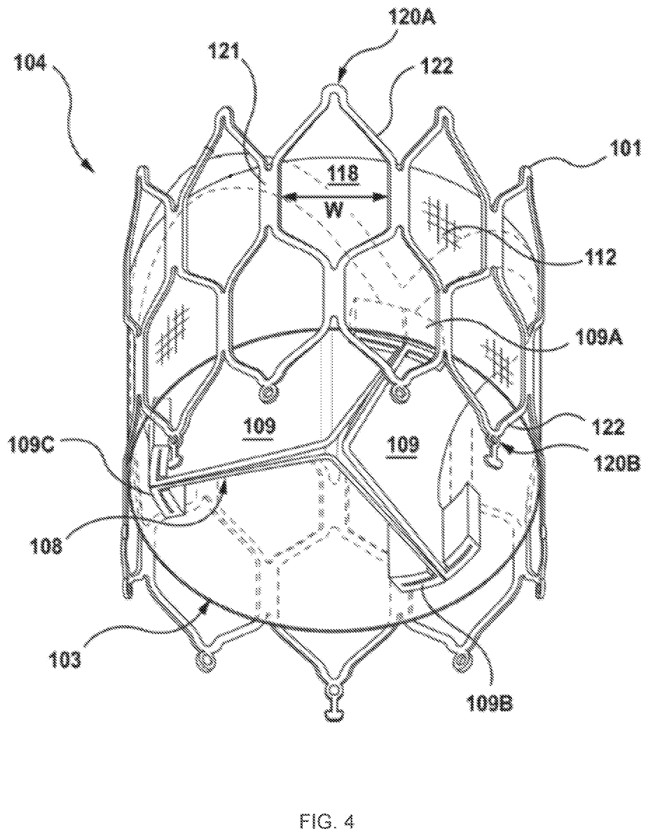

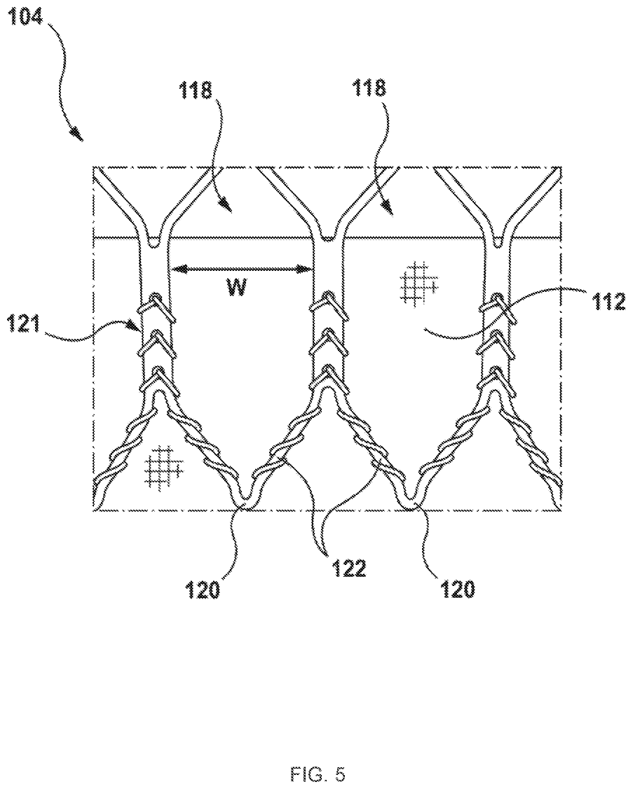

Referring to FIGS. 4 and 5, the inner frame 104 includes a plurality of side openings 118. The inner frame 104 may include a row of side openings 118 around a circumference thereof. The row of side openings may include less than twelve side openings, for example fewer than ten side openings. In some embodiments, the inner frame 104 may include three to eleven side openings, four to ten side openings, or six to nine side openings in the row.

The inner frame 104 includes a plurality of crowns 120 and a plurality of struts 122 with each crown 120 being formed between a pair of opposing struts 122. Each crown 120 is a curved segment or bend extending between opposing struts 122. The plurality of side openings 118 are defined by edges of the plurality of crowns 120 and the plurality of struts 122. In an embodiment, the plurality of side openings 118 may be substantially diamond-shaped. The inner frame 104 includes a plurality of nodes 121. A node 121 is defined as a region where two crowns of the plurality of crowns 120 within the inner frame 104 meet or connect.

The inner skirt 112 is attached to an inner surface of the inner frame 104 around a circumference thereof. The inner skirt 112 spans across or extends over a plurality of side openings 118 of the plurality of side openings 118. Notably, as is illustrated, it is not required that the inner skirt 112 extend over the full opening of each side opening 118 and/or that the inner skirt 112 extend over each of the plurality of openings. Rather, the inner skirt 112 may span or cover only a portion of the side opening 118.

A series of endmost inflow crowns 120A are formed at the inflow end 101 of the inner frame 104. A series of endmost outflow crowns 120B are formed at the outflow end 103 of the inner frame 104. In an embodiment, the inflow end 101 of the inner frame 104 has a total of nine endmost inflow crowns 120A around a circumference thereof. The outflow end 103 of the inner frame 104 has a total of nine endmost inflow crowns 120B around a circumference thereof. The inflow end 101 of the inner frame 104 includes a row of side openings 118 around a circumference thereof, and the row has a total of nine side openings 118. The outflow end 103 of the inner frame 104 includes a row of side openings 118 around a circumference thereof, and the row has a total of nine side openings 118. In another embodiment hereof (not shown), each of the inflow end 101 and the outflow end 103 of the inner frame 104 has between three and eleven, four to ten side openings, or six to nine side openings. In some embodiments, only a single row of side openings may be provided, or three or more rows of side openings may be provided.

In an embodiment, width W a width W of the side openings 118 is between 1/24 and ⅙ of the circumference of the inner frame 104, or stated another way, between 4% and 16% of the circumference of the inner frame 104. By increasing the width of the side openings 118, a lesser amount of material is required for the inner frame 104 such that a lower profile may be achieved when the inner frame 104 is crimped into a radially compressed configuration for delivery. For example, the dual frame 102 of some embodiments includes both the inner frame 104 and the outer frame 106 compressed around a common length. It can be a challenge to reduce the profile of the transcatheter valve prosthesis 100 in the crimped or radially compressed configuration in these and other embodiments. A challenge with reducing the profile of any such embodiments is that, in the crimped or radially compressed configuration, the incompressible material of the frame 102 imparts high compressive forces on the soft tissue material of the leaflets 109. Compressive forces may alter the integrity of the leaflets 109, thereby impacting the long-term durability of the transcatheter valve prosthesis 100. However, increasing the width W of the side openings 118 provides a reduction of the incompressible material of the dual frame 102, thereby enabling a lower profile in the crimped or radially compressed configuration.

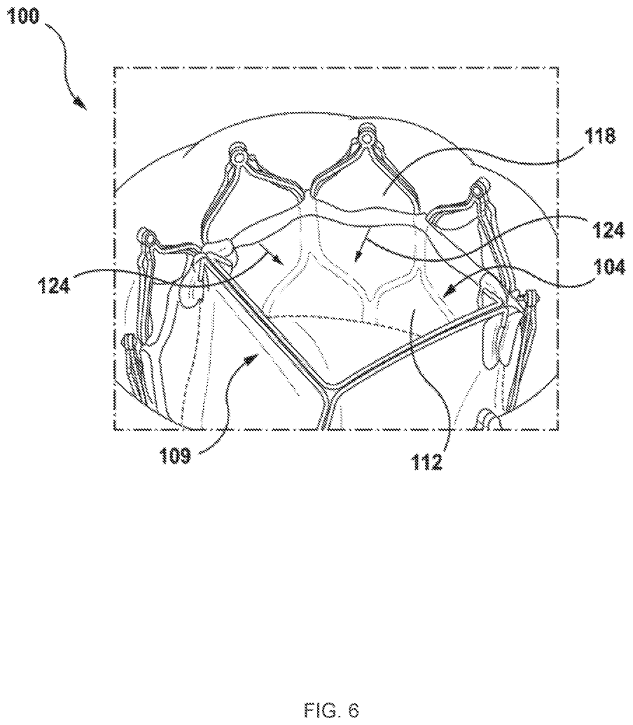

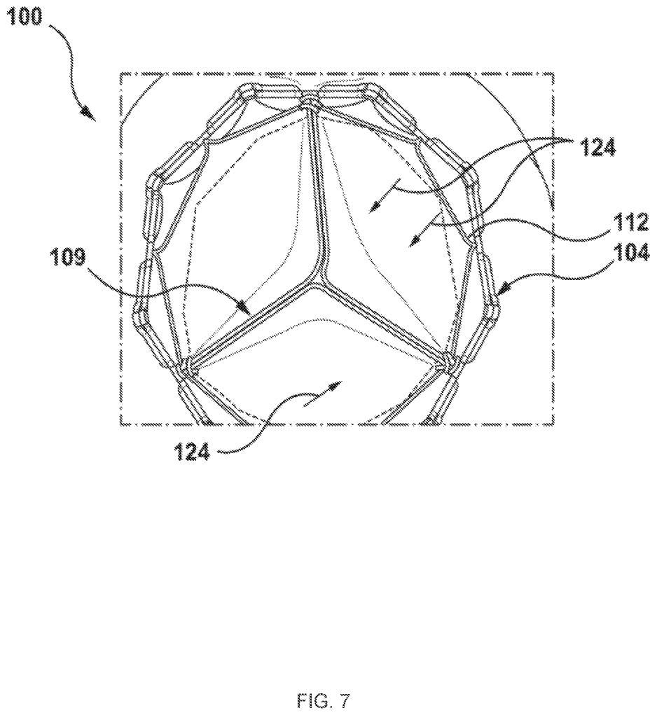

Reducing the incompressible material of the frame 102 means that the inner skirt 112 spans a longer distance between nodes 121 or between struts 122. Stated another way, increasing the width of the side openings 118 increases the amount of material of the inner skirt 112 that spans across the side openings 118. Thus, a greater amount of material of the inner skirt 112 is unattached to or unsupported by the inner frame 104. Referring now to FIGS. 6A and 6B, when an increased amount of material of the inner skirt 112 spans across the side openings 118, there is an increased chance of the inner skirt 112 billowing or moving radially inwards towards the leaflets 109 as indicated by directional arrows 124. The inner skirt 112 may billow during valve opening and closing in situ, and the leaflets 109 may contact the inner skirt 112. Such billowing may undesirably result in contact between the inner skirt 112 and the leaflets 109 of the transcatheter valve prosthesis 100. If the leaflets 109 of the transcatheter valve prosthesis 100 contact the inner skirt 112 during opening and closing, such contact may cause leaflet tissue abrasion as well as skirt abrasion. Additionally, the greater relative motion between the inner skirt 112 and the inner frame 104 may further induce skirt abrasion.

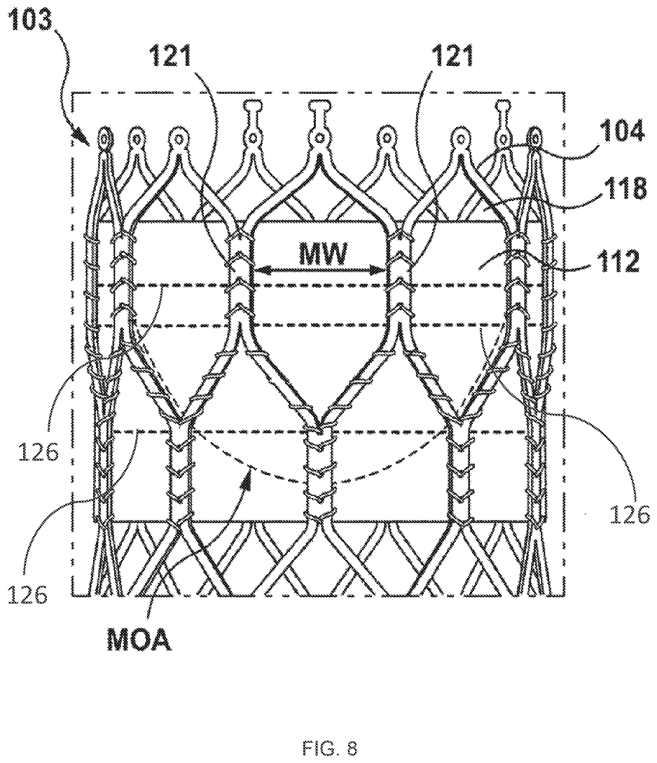

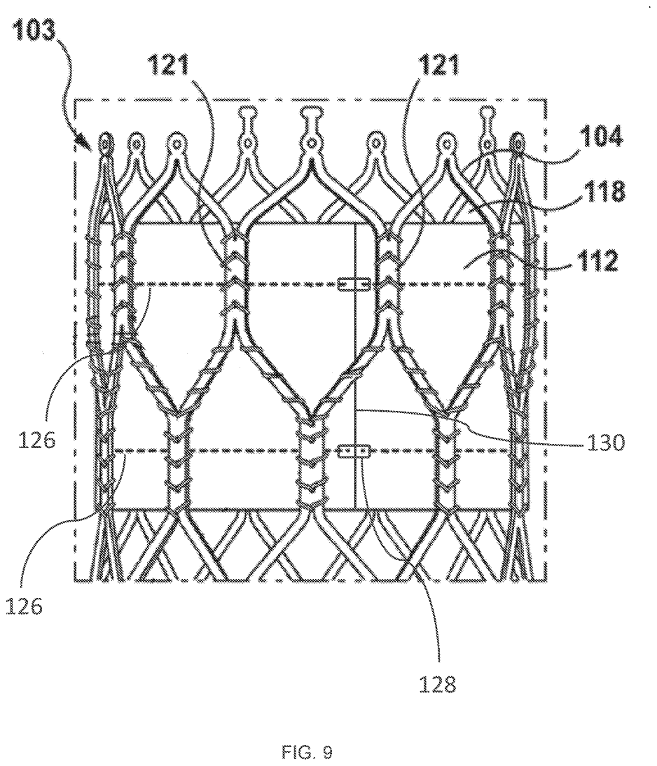

Referring now to FIGS. 8 and 9, the prosthesis 100 includes a skirt-reinforcement arrangement configured to prevent or restrict billowing of an area of the inner skirt 112 extending over, or at least partially covering, at least one side opening of the inner frame 104. Put another way, the skirt-reinforcement arrangement is configured to limit the radial motion of the skirt material. In this way, the skirt-reinforcing arrangement minimizes risk of damage to both the inner skirt 112 and the leaflets 109.

The skirt-reinforcement arrangement includes at least one reinforcement member 126 embedded within the inner skirt 112. In embodiments where the inner skirt 112 is formed from a fabric material, the at least one reinforcement member may be weaved into the fabric material or braided into the fabric material. Put another way, in some embodiments, the fabric material may be considered to be at least partially formed from the reinforcement members 126. The reinforcing member 126 may be considered to be a reinforcing wire that is embedded within the inner skirt 112.

The at least one reinforcing member 126 may include a plurality of reinforcing members 126. Embedding the reinforcement member 126 within the inner skirt enables the inner skirt to be reinforced without requiring the reinforcing member 126 to be connected to the inner frame 104. However, in alternative embodiments it will be appreciated that the at least one reinforcing member 126 may be connected to the inner frame 104.

The at least one reinforcement member 126 may extend around the inner skirt 112 such that it extends across a plurality of side openings 118. In some embodiments, the at least one reinforcement member 126 may extend at least partially around all of the side openings 118 in a particular row on the inner frame 104. The at least one reinforcement member 126 may extend around a minority (i.e. less than half) or a majority (i.e. more than half) of the inner skirt 112, for example substantially around all of the inner skirt 112.

In some embodiments, the fabric inner skirt 112 may be formed as a woven sheet arranged to be substantially tubular. Adjacent edges 130 of the fabric skirt may be attached, e.g. via stitching of an adhesive, to form a tubular skirt. In such embodiments, the at least one reinforcement member 126 may extend circumferentially around the inner skirt 112. In order to form the circumferential reinforcing ring, opposite or adjacent ends of the or each reinforcement member 126 are connected together. These connections may be made via a connecting member 128, for example via crimping. It will be appreciated that any suitable connection member 128 or connection arrangement may be used. In alternative embodiments, for example, the skirt 112 may be formed as a substantially cylindrical knitted tube.

As has been discussed above, the inner skirt 112 may be substantially tubular. In such embodiments, the at least one reinforcement member 126 may extend circumferentially around the inner skirt 112. In some embodiments, the at least one reinforcement member 126 may spiral around the inner skirt 112. In some embodiments, the at least one reinforcing member 126 may include a plurality of reinforcement members arranged as an array of overlapping reinforcement members 126 to form a reinforcing mesh.

In some embodiments, the at least one reinforcement member 126 may include at least one reinforcement member 126 extending along a direction between the inflow and outflow ends of the inner skirt 112. Put another way, the at least one reinforcement member 126 may include at least one reinforcement member 126 extending substantially perpendicularly to the at least one circumferential reinforcement member 126.

In some embodiments, the skirt-reinforcement arrangement is provided in the form of a plurality of spaced apart circumferential reinforcing rings, e.g. extending around an entirety of the inner skirt.

It will be appreciated that in some arrangements, a plurality of reinforcing rings may be formed by a single elongate reinforcing member 126, which extends around the inner skirt 112 in a first direction before folding back on itself to extend around the inner skirt 112 once more. Alternatively, each reinforcing ring may be formed by a separate reinforcing member 126. As has been discussed above, opposite or adjacent ends of the or each reinforcement member 126 are connected together. These connections may be made via a connecting member 128, for example via crimping. It will be appreciated that any suitable connection member 128 or connection arrangement may be used.

The at least one reinforcement member 126 may be configured to have a higher rigidity than a material forming the inner skirt 112. In some embodiments, the at least one reinforcement member 126 may be formed from a shape memory material, for example Nitinol. The reinforcement member may comprise one or more of: stainless steel, PET, PTFE, cobalt-chromium alloy, ceramic, polyvinylchloride (PVC), polyethylene (PE), polypropylene (PP), polymethylmethacrylate (PMMA), titanium, and/or polyester material(s), or any other biocompatible material(s) or combination of the above or other biocompatible materials, such as a hybrid PET+nitinol wire material.

It will be appreciated that the skirt-reinforcement arrangement described herein may be incorporated onto any prosthesis (i.e., a prothesis having a single frame or an inner frame and an outer frame coupled to and radially surrounding the inner frame) having an inner skirt for which reinforcement thereof is desirable to limit billowing, and it is not required that the stent or frame include a prosthetic valve component disposed therein.

A prosthesis having a radially expanded configuration and a radially compressed configuration, the prosthesis comprising: a frame comprising a plurality of side openings; a skirt coupled to the frame and at least partially covering at least one side opening of the plurality of side openings; and a skirt-reinforcement arrangement configured to prevent or restrict billowing of an area of the skirt extending over the at least one side opening, wherein the skirt-reinforcement arrangement comprises at least one reinforcement member embedded within the skirt.

The prosthesis according to paragraph A, wherein the skirt is substantially tubular, and wherein the at least one reinforcement member extends around a majority of the skirt.

The prosthesis according to paragraph B, wherein the at least one reinforcement member spirals around the skirt.

The prosthesis according to paragraph A, wherein the frame comprises a row of side openings extending around a perimeter thereof, and wherein the at least one reinforcement member extends across a plurality of side openings in the row of side openings, optionally wherein the at least one reinforcement member at least partially extends across each of the plurality of side openings in the row of side openings.

The prosthesis according to paragraph A, wherein the at least one reinforcement member extends circumferentially around the skirt.

The prosthesis according to paragraph E, wherein opposite ends of the reinforcement member are connected together to form a circumferential reinforcing ring extending around an entirety of the skirt.

The prosthesis according to paragraph F, wherein the opposite ends or edges of the reinforcement member are connected together via crimping.

The prosthesis according to paragraph F, wherein the skirt-reinforcement arrangement comprises a plurality of spaced apart circumferential reinforcing rings.

The prosthesis according to paragraph A, wherein the skirt-reinforcing arrangement comprise an array of overlapping reinforcement members arranged to form a reinforcing mesh.

The prosthesis according to paragraph A, wherein the skirt comprises a fabric material.

The prosthesis according to paragraph J, wherein the skirt is formed as a braided fabric, formed from a polymeric skirt material and a plurality of reinforcement members.

The prosthesis according to paragraph J, wherein the fabric material comprises a polymeric skirt material.

The prosthesis according to paragraph L, wherein the polymeric skirt material comprises one or more of PET, PTFE, and polyester.

The prosthesis according to paragraph A, wherein the at least one reinforcement member comprises a shape memory material.

The prosthesis according to paragraph N, wherein the shape memory material comprises nitinol.

The prosthesis according to paragraph A, wherein the at least one reinforcement member is configured to have a higher rigidity than a material forming the skirt.

The prosthesis according to paragraph A, wherein the reinforcement member comprises one or more of: stainless steel, PET, PTFE, nitinol, cobalt-chromium alloy, ceramic, polyvinylchloride (PVC), polyethylene (PE), polypropylene (PP), polymethylmethacrylate (PMMA), titanium, and/or polyester material(s), or any combination thereof.

The prosthesis according to paragraph A, wherein the skirt comprises a coat of a surface coating material.

The prosthesis according to paragraph R, wherein the surface coating material comprises one or more of: PTFE; polyurethane; silicone; and hydrogels.

The prosthesis according to paragraph A, wherein the at least one side opening of the plurality of side openings is substantially diamond-shaped.

The prosthesis according to paragraph T, wherein each of the plurality of side openings is substantially diamond-shaped.

The prosthesis according to paragraph A, wherein the frame comprises a row of side openings extending around a perimeter thereof, and wherein the row comprises fewer than twelve side openings, optionally fewer than ten side openings, for example in the range four to ten side openings, for example in the range six to nine side openings.

The prosthesis according to paragraph A, wherein the frame comprises a plurality of crowns and a plurality of struts with each crown being formed between a pair of opposing struts, and wherein the plurality of side openings are defined by the plurality of crowns and the plurality of struts.

The prosthesis according to paragraph A, wherein the skirt is coupled to an inner surface or an outer surface of the frame, and wherein the skirt extends over fewer than all of the plurality of side openings of the frame.

The prosthesis according to paragraph A, wherein the frame is formed from a self-expanding material.

The prosthesis according to paragraph Y, wherein the self-expanding material comprises a shape memory material, for example nitinol.
AA. The prosthesis according to paragraph A, wherein the frame comprises an inner frame and an outer frame, and wherein the plurality of side openings comprises a plurality of side openings on the inner frame and the skirt comprises an inner skirt coupled to the inner frame.
AB. The prosthesis according to paragraph AA, wherein the outer frame is connected to the inner frame and radially surrounds the inner frame, when the prosthesis is in the radially expanded configuration.
AC. The prosthesis according to paragraph AA, wherein the plurality of side openings comprises a plurality of side openings on the outer frame and the skirt comprises an outer skirt coupled to the outer frame.
AD. The prosthesis according to paragraph A, wherein the prosthesis is a heart valve prosthesis comprising a prosthetic valve component disposed within and secured to the frame, the prosthetic valve being configured to prevent blood flow in one direction to convey blood flow through a central lumen of the frame.
AE. The prosthesis according to paragraph AD, wherein the heart valve prosthesis is configured for placement within a mitral heart valve or tricuspid heart valve in situ.

Although the teachings have been described above with reference to one or more preferred embodiments, it will be appreciated that various changes or modifications may be made without departing from the scope as defined in the appended claims.

What is claimed is:

1. A prosthesis having a radially expanded configuration and a radially compressed configuration, the prosthesis comprising:
a frame comprising a plurality of side openings;
a skirt coupled to the frame and at least partially covering at least one side opening of the plurality of side openings; and
a skirt-reinforcement arrangement configured to prevent or restrict billowing of an area of the skirt extending over the at least one side opening, wherein the skirt-reinforcement arrangement comprises at least one reinforcement member embedded within the skirt.

2. The prosthesis according to claim 1, wherein the skirt is substantially tubular, and wherein the at least one reinforcement member extends around a majority of the skirt.

3. The prosthesis according to claim 2, wherein the at least one reinforcement member spirals around the skirt.

4. The prosthesis according to claim 1, wherein the frame comprises a row of side openings extending around a perimeter thereof, and wherein the at least one reinforcement member extends across a plurality of side openings in the row of side openings, optionally wherein the at least one reinforcement member at least partially extends across each of the plurality of side openings in the row of side openings.

5. The prosthesis according to claim 1, wherein the at least one reinforcement member extends circumferentially around the skirt.

6. The prosthesis according to claim 5, wherein opposite ends of the reinforcement member are connected together to form a circumferential reinforcing ring extending around an entirety of the skirt.

7. The prosthesis according to claim 6, wherein the opposite ends or edges of the reinforcement member are connected together via crimping.

8. The prosthesis according to claim 6, wherein the skirt-reinforcement arrangement comprises a plurality of spaced apart circumferential reinforcing rings.

9. The prosthesis according to claim 1, wherein the skirt-reinforcing arrangement comprise an array of overlapping reinforcement members arranged to form a reinforcing mesh.

10. The prosthesis according to claim 1, wherein the skirt is formed as a braided fabric, formed from a polymeric skirt material and a plurality of reinforcement members.

11. The prosthesis according to claim 1, wherein the at least one reinforcement member comprises a shape memory material.

12. The prosthesis according to claim 1, wherein the at least one reinforcement member is configured to have a higher rigidity than a material forming the skirt.

13. The prosthesis according to claim 1, wherein the at least one side opening of the plurality of side openings is substantially diamond-shaped.

14. The prosthesis according to claim 1, wherein the frame comprises a row of side openings extending around a perimeter thereof, and wherein the row comprises fewer than twelve side openings, optionally fewer than ten side openings, for example in the range four to ten side openings, for example in the range six to nine side openings.

15. The prosthesis according to claim 1, wherein the frame comprises a plurality of crowns and a plurality of struts with each crown being formed between a pair of opposing struts, and wherein the plurality of side openings are defined by the plurality of crowns and the plurality of struts.

16. The prosthesis according to claim 1, wherein the skirt is coupled to an inner surface or an outer surface of the frame, and wherein the skirt extends over fewer than all of the plurality of side openings of the frame.

17. The prosthesis according to claim 1, wherein the frame comprises an inner frame and an outer frame, and wherein the plurality of side openings comprises a plurality of side openings on the inner frame and the skirt comprises an inner skirt coupled to the inner frame.

18. The prosthesis according to claim 17, wherein the outer frame is connected to the inner frame and radially surrounds the inner frame, when the prosthesis is in the radially expanded configuration.

19. The prosthesis according to claim 17, wherein the plurality of side openings comprises a plurality of side openings on the outer frame and the skirt comprises an outer skirt coupled to the outer frame.

20. The prosthesis according to claim 1, wherein the prosthesis is a heart valve prosthesis comprising a prosthetic valve component disposed within and secured to the frame, the prosthetic valve being configured to prevent blood flow in one direction to convey blood flow through a central lumen of the frame.
